(19) United States
(12) Patent Application Publication
(10) Pub. No.: US 2019/0298621 A1
(43) Pub. Date: Oct. 3, 2019

(54) METHODS OF TREATMENT OF THE DENTAL PULP AND FILLING ROOT CANALS USING ANTI INFLAMMATORY RINSE SOLUTION AND FILLING COMPOSITION

(71) Applicant: DENTSPLY SIRONA Inc., York, PA (US)

(72) Inventor: Todd Patrick BERGER, Owasso, OK (US)

(73) Assignee: DENTSPLY SIRONA Inc., York, PA (US)

(21) Appl. No.: 16/365,859

(22) Filed: Mar. 27, 2019

Related U.S. Application Data
(60) Provisional application No. 62/648,614, filed on Mar. 27, 2018.

(51) Int. Cl. A61K 6/00

(52) U.S. Cl. CPC A61K 6/0067; A61K 6/0035; A61K 31/355; A61K 6/0091; A61K 6/0038

(57) ABSTRACT

A method of reducing an inflammation of a pulp or a periodontal ligament, said method comprising the steps of: (a) removing material from a pulp cavity and/or a root canal of a tooth; (b) rinsing pulp chamber with an anti-inflammatory rinse solution, (c) irrigating the root canal with an anti-inflammatory rinse solution to remove residual bacteria and/or smear layer, (d) providing an anti-inflammatory filling composition comprising: (i) at least 80% by weight of a premixed cement putty, said cement putty including at least one calcium silicate compound in the range of about 30% to about 90% by weight of said putty; and at least about 1% to about 50% of a liquid carrier including a water-soluble polymer and/or water-soluble oil; and (ii) about 3 to about 15% by weight of an anti-inflammatory agent, the anti-inflammatory agent including eugenol, vitamin E or mixtures thereof, and (e) introducing the anti-inflammatory filling composition into the cavity and/or root canal and allow the composition to harden.

### RELATED APPLICATIONS

This patent application claims the benefit of and priority to U.S. Provisional Application Ser. No. 62/648,614, filed on Mar. 27, 2018, which is herein incorporated by reference for all purposes.

### FIELD OF THE INVENTION

The present invention relates to methods and compositions for reducing the inflammation of a pulp or a periodontal ligament by way of providing an anti-inflammatory rinse solution and/or an anti-inflammatory filling composition to the pulp chamber and/or the root canal.

### BACKGROUND OF THE INVENTION

The inner portion of a tooth includes a pulp cavity that contains soft living tissue or the “pulp” of the tooth. The pulp includes connective tissue, blood vessels, cells, and nerve endings. The pulp cavity comprises an upper pulp chamber and root canals that extend to the apex or apical section of the tooth deeper into the jaw. The outer (visible) portion of the tooth is referred to as the crown and has a covering of enamel. The hard enamel protects softer dentinal tissues in the upper portion of the tooth. The enamel consists of a hard, calcium-based substance, hydroxyapatite. The dentin tissue contains a matrix of minute tubules interspersed with collagen fibers that surround and protect the tooth pulp. The outer (non-visible) portion of the tooth root is covered with cementum, a thin hard tissue that joins the root to the surrounding bone through Sharpey's fibers. Dental decay, or caries, is caused by bacteria accumulating on teeth and forming a biofilm (plaque). The biofilm produces acids that dissolve and weaken the hydroxyapatite of the tooth, thereby causing decay.

While various chemical and physical irritants can cause irritation and even necrosis of the pulp, the most common causes for pulpal inflammation (pulpitis) are bacteria and/or their products entering the pulp through a deep caries lesion or a leaking filling; an inflammatory reaction in the pulp starts long before bacteria invade the pulp tissue. The inflammatory reaction is first initiated by bacterial antigens interacting with the local immune system. As long as the carious lesion has not entered the pulp, the pulpal inflammation is likely to be reversible. However, when the carious lesion does reach the pulp and the hard tissue barrier is breached, bacteria can invade the pulp. Even after this point, the infection may remain relatively superficial and most of the pulp tissue is vital and bacteria free. For this reason, endodontic treatment of pulpitis should be considered to be treatment of an inflammation and prevention of an infection.

In apical periodontitis, bacteria invade further and colonize the entire root canal system. Apical periodontitis is an inflammatory process in the periradicular tissues caused by microorganisms in the necrotic root canal. Accordingly, to promote healing of apical periodontitis, microorganisms within the root canal system must be eliminated. Provided that the dental disease is not too progressed, dental professionals will use root canal treatment procedures to remove the infected tissue from the tooth and replace it with an inert, biocompatible material. Otherwise, extraction of the tooth might be required.

The root canal system of a tooth is complex and many treatment methods can be used depending upon the condition of the patient and approach of the practitioner. In general, root canal treatment methods first involve drilling an opening in the crown of the tooth to provide access to the pulp cavity. Then, endodontic files are used to remove the pulp and clean and shape the root canals. The files are used with an irrigant. After using the files, an irrigant may be used to remove the smear layer created by the files. Irrigation plays the main role in eradication of microbes from the root canal system.

Locally used endodontic disinfectants, either irrigating solutions or interappointment medicaments, are effective against a wide spectrum of microorganisms. For example, hypochlorite solution is effective against bacteria and yeast; even bacterial spores are killed with high concentration (5%) sodium hypochlorite. Hypochlorite has the ability to dissolve organic debris (for example necrotic pulp tissue), however, hypochlorite cannot remove smear layer that has formed on canal walls that have been in contact with rotary preparation instruments. Smear layer has traditionally been removed by 17% EDTA (ethylenediaminetetraacetic acid), and citric acid has also been used for this purpose. Recently, QMix™ 2in1 a premixed, ready-to-use, colorless and odorless solution that is free of antibiotics has proven to be a highly effective antimicrobial agent (99.99% disinfection per independent studies).

There is continuing need of product that reduces the inflammation of an inflamed pulp or inflamed periodontal ligament after the shaping by instrument, high speed burr or coronal pulp amputation.

It is an object of the invention to provide anti-inflammatory rinse to be applied into the root canal or pulp chamber to reduce inflammation of the associated tissues.

Optionally, a sealer is coated on the wall of the root canals prior to the root canals being filled with a filling material. This sealing of the roots ideally prevents bacteria and other microorganisms from re-entering and causing infection of the living tissue surrounding the root tip. In a final step, the pulp chamber and opening in the crown of the tooth is sealed with a dental restoration such as a filling material. Preferably a permanent crown is placed over the opening in the tooth, such crowns being made of metal, porcelain-enameled metal, polymer-veneered metal, or ceramic. A post may be placed in the root for stability of the crown, during the filling step of the root canal procedure, before the crown is applied.

One method for filling root canals involves using naturally occurring or synthetic gutta-percha, an isomer of rubber. Historically, one older treatment method involves using single cones of gutta-percha. In this method, zinc oxide-eugenol cement sealer is first placed in the root canal. Then a single unheated cone of gutta-percha is fitted into the root canal.

Other root canal treatment methods involve using portland cement to repair root defects such as iatrogenic perforations, or when apical surgery is performed to fill the root end. In general, portland cement contains a compound formed from calcia, silica, alumina, and iron oxide materials. Portland cement is commonly gray, but white versions, with lower iron content are known. The portland cement is combined with water to form a slurry-like composition that is introduced into the root canal defect. The composition solidifies to seal the canal. When portland cement materials are used to fill or seal the root canals, the cement particulates should have a small particle size. The fineness of a cement is represented by the surface area and one measurement thereof is the Blaine Number representing the ratio of the cement's particle surface area to its weight (square centimeters of surface per gram).

Torabinejad et al., U.S. Pat. Nos. 5,769,638 and 5,415,547 describe using a portland cement composition. In addition to portland cements, other biomedical cements have been developed for medical and dental applications. For example, Lu et al., US Patent Application Publication US 2007/0098811 discloses a biomedical cement containing at least one phosphate compound and at least one calcium silicate compound that does not contain any aluminum or magnesium compounds. Yang and Lu, U.S. Pat. No. 8,475,811 discloses premixed cement paste for use in medical or dental application. Asgary, U. S. Pat. Nos. 7,942,961 and 8,105,086 discloses bioactive endodontic material and its use for filling the tooth and bone cavities.

Another material that is used in surgical and non-surgical root canal procedures is ProRoot™ MTA root repair material available from Dentsply Tulsa Dental Specialties (Tulsa, Okla.). ProRoot MTA material has a composition similar to portland cement and does not contain any water-soluble polymeric materials. Particularly, the MTA material includes fine hydrophilic particles of dicalcium silicate, tricalcium silicate, tricalcium aluminate, tetracalcium aluminoferrite, calcium sulfate dihydrate, and bismuth oxide that are combined with water to form a cement-like material. The MTA material is available in gray and white colored formulations. The oxides used in the MTA powder are of the highest purity to ensure that no heavy metals are included and used in the body. MTA root canal repair material is used in a wide variety of clinical applications. Particularly, the cement-like material has been used to repair root canal perforations during root canal therapy; fill root ends; treat injured pulps in procedures known as pulp capping and pulpotomy, and repair root resorption.

MTA products are now expanding to restoration (Luting cements) products. All of these applications include the possibility of the patient having an inflamed pulp or inflamed periodontal ligament. This is what causes the patient discomfort before and after a procedure by the clinician. In some cases, the patient calls the clinician unexpectedly some hours after the procedure to complain about the discomfort. There is continuing need to minimize the patient discomfort. By including an anti-inflammatory in an MTA product all the indications of use would then minimize the post procedure discomfort of the patient. It is expected that once the material is placed (before and after setting), the anti-inflammatory additive would have to be released or be on the surface in sufficient quantity to reduce the inflammation such that the patient's discomfort has been prevented.

The present invention provides such anti-inflammatory rinse solution and filling materials.

### SUMMARY OF THE INVENTION

The present disclosure provides compositions for reducing inflammation of an inflamed pulp or inflamed periodontal ligament, after the shaping by instrument, high speed burr or coronal pulp amputation.

In a first aspect of the present disclosure there is provided an anti-inflammatory rinse solution for removing the smear layer and disinfection in a root canal or pulp chamber. In a second aspect of the present disclosure there is provided an anti-inflammatory endodontic filling composition comprising: (a) at least 80 percent by weight of a premixed cement putty, said cement putty including at least one calcium silicate compound in the range of about 30% to about 90% by weight of said putty; and at least about 1% to about 50% of a liquid carrier including a water soluble polymer and/or water soluble oil; and (b) about 1 to about 20% by weight of an anti-inflammatory agent including Eugenol, Vitamin E, or a mixture thereof.

In a third aspect of present disclosure there is provided a method of reducing an inflammation of a pulp or a periodontal ligament, said method comprising the steps of:

(a) removing material from a pulp cavity and/or a root canal of a tooth;
(b) rinsing the pulp cavity with an anti-inflammatory rinse solution;
(c) irrigating the root canal with the anti-inflammatory rinse solution to remove residual bacteria and/or smear layer;
(d) providing an anti-inflammatory filling composition comprising:

(i) at least 80 percent by weight of a premixed cement putty, said cement putty including at least one calcium silicate compound in the range of about 30% to about 90% by weight of said putty; and at least about 1% to about 50% of a liquid carrier including a water-soluble polymer and/or water-soluble oil; and

(ii) about 3 to about 15% by weight of an anti-inflammatory agent, the anti-inflammatory agent including eugenol, vitamin E or mixtures thereof.

(e) introducing the anti-inflammatory filling composition into the cavity and/or root canal and allow the composition to harden.

Other aspects will be set forth in the description which follows, and in part will be apparent from the description or may be learnt by the practice of the invention.

### BRIEF DESCRIPTION OF THE FIGURES

FIG. 1. Molecular structure of Eugenol, which is partially soluble in water.

FIG. 2. Molecular structure of Vitamin E, which is insoluble in water.

FIG. 3. Molecular structure of D-α-Tocopherol polyethylene glycol 1000 succinate, which is soluble in water.

FIG. 4. Molecular structure of Ibuprofen; a reference anti-inflammatory, which is partially soluble in water.

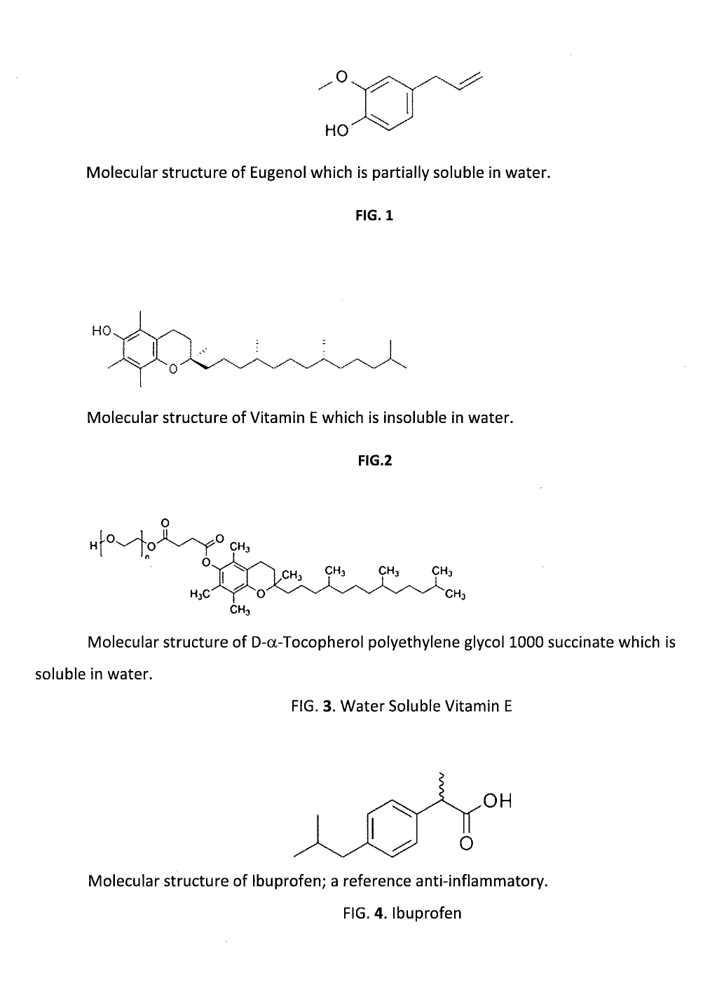

### DETAILED DESCRIPTION OF THE INVENTION

The above-mentioned aspects, as well as other aspects, features, and advantages of the present technology is described below in connection with various embodiments, with reference made to the accompanying drawings

Some of the terms used in the present disclosure are defined below:

The term “Anti-inflammatory” as used herein, is counteracting inflammation or reduces the inflammation of inflamed tissue (pulp or periodontal ligament) such that it minimizes patient discomfort.

The term “smear layer” as used herein, is well known to persons skilled in the art of dentistry and refers to the complex accumulation of organic and inorganic debris resulting from the mechanical preparation of a tooth surface. The smear layer comprises cutting debris, tooth particles, microorganisms, necrotic material, and other substances resulting from preparation, and typically includes a superficial layer on the surface of a prepared tooth along with a layer or layers that are packed into the adjacent dentinal tubules at varying depths up to about 40 μm. In the context of orthopedics, “smear layer” refers to similar layers in prepared bone sites.

The present disclosure provides methods and compositions for reducing the inflammation of a pulp, root canal and/or a periodontal ligament. The method includes providing an anti-inflammatory rinse solution for removing smear and disinfection in a root canal or pulp chamber, and/or introducing an anti-inflammatory filling composition to the pulp chamber and the root canal.

In certain embodiments of the anti-inflammatory rinse solution disclosed herein includes at least one anti-inflammatory agent.

In certain embodiments of the anti-inflammatory rinse solution, the anti-inflammatory agent is selected from the group consisting of tannoid, benzydamine, vitamin E, eugenol, saline water, phosphate buffer saline, natural phytochemical antioxidant, chalcone, curcuminoid, carotenoid, Sambucus nigra, Echinacea purpurea, Hydrocotyle asiatica, xylitol and a mixture thereof.

In certain embodiments of the anti-inflammatory rinse solution, the anti-inflammatory agent is eugenol.

In certain embodiments of the anti-inflammatory rinse solution, the anti-inflammatory agent is present in an amount of less than 25% w/w, preferably less than 20% w/w or the anti-inflammatory agent is present in an amount of at least 2%, preferably at least 5% w/w. In certain embodiments of the anti-inflammatory rinse solution, the anti-inflammatory agent is present in concentration range from about 2 to about 25% w/w, preferably about 5 to about 20% w/w.

In certain embodiments of the anti-inflammatory rinse solution, the anti-inflammatory rinse solution further includes a mixture of chlorhexidine and EDTA. Chlorhexidine may be present in the mixture in a concentration of at least about 0.1% by weight and up to about 5.0% by weight, with the most preferable being about 1% to about 3.5% (e.g., about 2%). EDTA may be present in the mixture in an amount from about 0.5 to about 30% by weight, more preferably EDTA is present in an amount from about 2 to about 25%, with the most preferable being about 10 to about 20% (e.g., about at least 17%). The total mixture of chlorhexidine and EDTA is present in a concentration of from about 50% to about 98%, more preferably in the range of 80% to 95%, more preferably at least 85%.

Also provided herein are anti-inflammatory filling compositions that may be used for cavity lining or pulp capping of carious teeth, treatment of traumatized teeth or any procedure where bacterial leakage is to be minimized between the coronal and apical areas.

The composition of the anti-inflammatory filling may be made from two parts: Part (a) of the composition may be a premixed cement putty. The cement putty may include at least one calcium silicate compound and a liquid carrier including a water-soluble polymer. Example of calcium silicate compound that may be used include but are not limited to, dicalcium silicate, tricalcium silicate and a mixture thereof. In certain embodiments of the anti-inflammatory filling composition, calcium silicate includes a mixture of tricalcium silicate and dicalcium silicate particles. The calcium silicates may be included in the range of about 20% to about 95% by weight in the putty composition, preferably in the range of about 30% to about 90% by weight of the putty composition.

The cement putty may further include a filler material. In some embodiments of anti-inflammatory endodontic filling composition, the filler material may include bioglass. In some embodiment, bioglass may include 40-62% by weight SiO₂; 10-32% by weight Na₂O; 12-35% by weight CaO, and 0-12% by weight P₂O₅. In some embodiments of anti-inflammatory endodontic filling composition, the bioglass may include 35-55% by weight SiO₂; 12-35% by weight Na₂O; 10-32% by, weight CaO, and 3-9% by weight P₂O₅. In some embodiments of anti-inflammatory endodontic filling composition, the filler material has particle size in the range of about 0.1 microns to about 400 microns. Preferably, in the range of about 0.25 microns to 120 microns. The filler may be present in concentration range of about 1 to about 40%, preferably in the range of about 3% to about 20%.

In some embodiments, the cement putty may further include a radiopacifier. Exemplary radiopacifier include calcium, strontium, zirconium, lanthanum, tungsten, bismuth or barium compounds; preferably zirconium or barium compounds; more preferably zirconium compounds. In some embodiments, the radiopacifier may be selected from strontium oxide, zirconium silicate, zirconium oxide, zirconium dioxide, lanthanum oxide, calcium tungstate, bismuth oxide, barium zirconate and barium sulphate or a combination thereof; more preferably zirconium dioxide and barium sulphate; most preferably zirconium dioxide.

In some embodiments, the cement putty may comprise from 0% to 50% radiopacifier, preferably from 10% to 40% radiopacifier; more preferably from 20% to 30% radiopacifier; most preferably about 25% radiopacifier.

Example of suitable liquid carriers include but are not limited to ethyl alcohol, alkylene glycol, poly(alkylene glycol), glycerin, 1-methyl-2-pyrrolidone, liquid organic acid, and mixtures thereof. In certain embodiments of anti-inflammatory endodontic filling composition, the liquid carrier is poly(alkylene glycol). Polyalkylene glycols are polymeric ethers and therefore come in a variety of different molecular weights. In certain embodiments of anti-inflammatory endodontic filling composition, the poly(alkylene glycol) is poly propylene glycol having a number average molecular weight in a range of from 1500 g/mol to 3000 g/mol. In some embodiments of anti-inflammatory endodontic filling composition, the composition comprises from 1% to 50% of the polyalkylene glycol; more preferably about 25%.

Example of water-soluble polymers include, but are not limited to, non-ionic polymers such as, for example, polyvinyl alcohols (PVA) and its co-polymers, partially hydrolyzed polyvinyl acetates, (PVAc), polyvinyl-pyrrolidone (PVP), hydroxyethyl methacrylate (HEMA) and water-soluble poly-saccharides (e.g. xanthan gum). Various water-soluble co-polymers containing the above residues also can be used. Additional examples of water-soluble polymers include anionic polymers such as, for example, polyacrylic acid (PAA), its water-soluble salts, derivative and copolymers, polymethacrylic acid (PMA) its water-soluble salts, derivatives and its water-soluble copolymers, water-soluble copolymers containing maleic acid residues, poly-glucuronic acid, poly-glutamic acid its water-soluble salts, poly-aspartic acid and its water-soluble salts, hyaluronic acid and its water-soluble salts and derivatives, polystyrene sulfonates its salts and their copolymers.

In a preferred embodiment, the water-soluble polymer is selected from the group consisting of polyvinyl alcohols, polyvinyl-pyrrolidone (PVP), polyvinyl acetates, and mixtures thereof. Preferably, the molecular weight of the water-soluble polymer is in the range of 20,000 to 2,000,000. More preferably, the molecular weight of the water-soluble polymer is in the range of 80,000 to 2,000,000. The water-soluble polymer may be present in concentration range of about 0.1 to about 10%, preferably in the range of about 1% to about 5%.

Example of water-soluble oil includes D-α-Tocopherol polyethylene glycol 1000 succinate. The water-soluble oil may be present in concentration range of about 0.1 to about 20%, preferably in the range of about 5% to about 15%, more preferably about 10%.

In certain embodiments of anti-inflammatory endodontic filling composition, the premixed cement putty is present in an amount of less than 95% w/w, preferably, less than 90% w/w or the premixed cement putty is present in an amount of at least 30%, preferably at least 65% w/w and most preferably at least 85% by weight based on the total composition.

In certain embodiments of anti-inflammatory endodontic filling composition, the premixed cement putty is present in the concentration range of about 30% to 95% by weight based on the total composition, preferably in the range of about 65% to 90% by weight

Part (b) of the composition may be an anti-inflammatory agent. The anti-inflammatory agent may be eugenol, vitamin E, or a mixture thereof. In certain embodiments of anti-inflammatory endodontic filling composition, the anti-inflammatory agent is present in an amount of less than 25% w/w, preferably less than 20% w/w or the anti-inflammatory agent is present in an amount of at least 1%, preferably at least 3% w/w.

In certain embodiments of anti-inflammatory endodontic filling composition, the anti-inflammatory agent may be present in concentration range of from 1% to 20% by weight based on the total composition, more preferably in the range of about 3% to 15%.

In practice the clinician may dispense the calcium silicates, add the liquid carrier; and mix the components together using a spatula to form cement putty. The concentration of calcium silicates in the composition is generally in the range of about 30% to about 90% by weight of the putty composition. To prepare a surgical or repair composition the particulate powder is preferably mixed with the liquid carrier in a ratio of three (3) to one (1). That is, in one preferred embodiment, the composition may include about 75 weight percent calcium silicate and 25 weight percent liquid carrier. In other instances, the particulate powder may be mixed with the liquid carrier in different ratios such as, for example, four (4) to one (1) or five (5) to one (1) or anywhere inbetween. If the composition is intended to be used as a root canal sealer, the powder and liquid are preferably mixed in a ratio in the range of 3:1 to 6:1. In the final composition, the water content may be generally in the range of about 1 to about 60 percent, preferably 5 to 50%, more preferably about 10-40% and even more preferably about 14 to 33%.

Upon mixing the calcium silicate with the liquid carrier, the particles, which are hydrophilic, react with the liquid to form hydrates. For example, the calcium silicate preferably contains particles of tricalcium silicate, and dicalcium silicate. When these compounds react with water, they produce tricalcium silicate hydrate, and dicalcium silicate hydrate. It is believed that each mineral compound reacts at a different rate. For example, the tricalcium silicate may react relatively quickly, while dicalcium silicate may hydrate more slowly. The material produced from the hydration reaction is a colloidal hydrate gel. Preferably, the particles dispersed in the gel have a very small particle size as discussed above. The product begins to harden and will eventually solidify to form a material having high compressive strength where the particles are mostly hydrated. Because the mixed material has good resistance to washout and displacement, the particulate material can react with the water and form a mass of relatively high compressive strength of from about 5 to 50 MPa, preferably from about 10 to 50 MPa, more preferably from about 15 to 30 MPa and most preferably about 24 MPa after 3 days. Preferably, the material is able to resist washing out when the root canal system is rinsed with water, or other fluid to complete a surgical procedure.

It should be understood that mixing the powdered material with a liquid carrier (having water-soluble polymer and/or water) as described above is but only one specific example of preparing the composition of this invention. Other methods may be used. For example, Part (a) may be prepared by blending the powdered particulate with the liquid carrier including water soluble polymer and Part (b) may be prepared by dissolving the polymer in water. Then, Parts (a) and (b) may be combined to form the composition that may be used in dental therapy. Another technique involves mixing the powdered particulate with water (Part a) and then combining this mixture with a previously prepared mixture of water-soluble polymer and/or surfactant (Part b).

The compositions of this invention provide enhanced bonding to gutta-percha and to dentin. Bonding of sealer to dentin or gutta-percha has been a topic of great concern to endodontists in the prevention of bacterial migration in obturated, root-canal-treated teeth. The hydrophilic nature of the calcium silicate enhances the reactivity of the composition of the present invention with moist dentin. In addition, the formulations of the present invention have enhanced bonding to gutta-percha, owing to the presence of the hydrophobic side chains in the partially hydrolyzed polyvinyl acetate and/or other polymers used. The hydrophobic side-chains have an affinity for gutta-percha. Because of their improved bonding properties, the composition provides an improved barrier to bacterial and fluid leakage in the root canal system of a tooth. The composition effectively seals offs communication pathways from the coronal to the apical portions when used as a root canal sealer, obturation material, root-end filling, apexification, perforation repair, or root resorption. As a result, bacterial migration into the root canal system is reduced or prevented.

The compositions of this invention may be either putty-like or syrupy in viscosity. When the composition is in the form of a putty-like material, it may be used in root canal indications such as apicoectomies, apexification, perforation repair, obturation, pulpotomies, or root-resorption repair. When the composition is in the form of an elastic material having a honey-like consistency, it may be used for root canal sealing or perhaps obturation. The rheological properties (viscosity, elasticity, and the like) of the powder-liquid combination are determined by the particle size distribution of the powder, the composition of the liquid, and the powder to liquid ratio. Finer powders; more viscous liquids; more polymers; and a higher powder to liquid ratio all make a more putty-like material used for pulp-capping, cavity liner, root-end filling, obturation, pulpotomies, apexification, or treating perforations or root resorption. The composition of this invention is introduced into the tooth from the coronal or apical openings.

For example, the compositions may be used to seal at least a portion of the tooth; repair root perforations; repair root resorption; fill root ends; and/or cap at least a portion of the dental pulp that has been exposed. The composition also may be used to line a cavity preparation where pulp-exposure is possible. Moreover, complete obturation of root canals may be performed using the material of this invention. In addition, after a pulpotomy has been performed, the composition may be used to cover a root access opening in a root. In yet another example, the composition may be used to seal a root canal after gutta-percha has been introduced into the canal.

In another embodiment, the present disclosure provides a method of reducing an inflammation of a pulp or a periodontal ligament. The method may involve removing material from a pulp cavity and/or a root canal of a tooth; rinsing pulp chamber with an anti-inflammatory rinse solution irrigating the root canal with an anti-inflammatory rinse solution of the present disclosure to remove residual bacteria and/or smear layer; providing an anti-inflammatory filling composition comprising: (a) about 30% to 95% by weight of a premixed cement putty, said cement putty including at least one calcium silicate compound in the range of about 30% to about 90% by weight of said putty; and at least about 1% to about 50% of a liquid carrier including a water soluble polymer and/or water soluble oil; and (b) about 3 to about 15% by weight of an anti-inflammatory agent, the anti-inflammatory agent including eugenol, vitamin E, or mixtures thereof; introducing the anti-inflammatory filling composition into the cavity and/or root canal and allow the composition to harden.

In certain embodiments of a method of reducing an inflammation of a pulp or a periodontal ligament, the anti-inflammatory filling composition may be provided wherein the premixed cement putty may be present in an amount of less than 95% w/w, preferably, less than 90% w/w or the anti-inflammatory agent may be present in an amount of at least 30%, preferably at least 65% w/w and most preferably at least 85% by weight based on the total composition.

In one embodiment of a method of reducing an inflammation of a pulp or a periodontal ligament, an anti-inflammatory filling composition may comprise: (a) at least 85% by weight of a premixed cement putty, said cement putty including at least one calcium silicate compound in the range of about 30% to about 90% by weight of said putty; and at least about 1% to about 50% of a liquid carrier including a water soluble polymer and/or water soluble oil; and (b) about 3 to about 15% by weight of an anti-inflammatory agent, the anti-inflammatory agent including eugenol, vitamin E or mixtures thereof.

The invention may further be illustrated by the compositions described in the following Examples, but these Examples should not be construed as limiting the scope of the invention.

### EXAMPLES

### Example 1: Anti-Inflammatory Rinse Solution

An anti-inflammatory rinse solution was prepared by mixing anti-inflammatory agent in a solvent in which they are miscible in a vial under ambient conditions as shown in Table 2.

TABLE 1
Anti-inflammatory agent solubility in solvents

| Anti-Inflammatory | Solvent | Group |
|---|---|---|
| Chalcone | Ethanol | A |
| α-tocopherol | Ethanol | A |
| Eugenol | Ethanol | A |
| Curcuminoid | Water | B |
| Carotenoid | Water | B |
| Sambucus nigra | Water | B |
| Echinacea purpurea | Water | B |
| Centella asiatica (hydrocutyle asiatica) | Water | B |
| Xylitol | Water | B |
| D-α-Tocopherol polyethylene glycol 1000 | Water | B |
| succinate | | |

TABLE 2
Anti-inflammatory rinse composition in different solvents

| Ingredient | Example A | Example B | Example C |
|---|---|---|---|
| Group A | 5% | | |
| Group B | | 5% | 5% of one choice from Group B |
| | | | and 5% of a second choice from |
| | | | Group B (example: 5% of |
| | | | Carotenoid & 5% of Xylitol) |
| Water | | 95% | 90% |
| Ethanol | 95% | | |
| Total | 100% | 100% | 100% |

### Example 2: Reference Example A

Ibuprofen (caplet form) was grounded in a mortar and pestle to a powder form. The powder was sieved to about 50 μm. The resulting powder was then blended with cement in a V blender in the following compositions mentioned in the Table 3 below.

TABLE 3
Reference Anti-Inflammatory Powder Formulations

| | ProRoot | |
|---|---|---|
| Ingredient | (powder) | Reference |
| Cement | 80% | 78.4% |
| Radiopacifier | 20% | 20.4% |
| Ibuprofen | | 1.3% |

The powder was mixed with water at a ratio from 3:1 to 6:1. Additional loading of ibuprofen was easily accomplished.

### Example 3: Using Eugenol: 1) Paste 2) Powder to be Mixed with Water

Eugenol was purchased as a liquid and was used to produce two prototypes:

Prototype #1—“Eugenol Paste”: Eugenol was dissolved in an oil then mixed in a speed mixer with other ingredients

Prototype #2—“ZOE Powder”: Eugenol was reacted with ZnO and allowed to set hard, the resulting hardened material was ground with a mortar and pestle. The resulting powder was then blended with cement in a V blender compositions mentioned in the Table 4 below.

TABLE 4
Examples of Eugenol Anti-Inflammatory MTA

| Ingredient | Paste | Ingredient | Powder |
|---|---|---|---|
| Cement | 61.2% | Cement | 98% |
| Radiopacifier | 19.4% | ZOE | 2% |
| Bioglass | 4.9% | | |
| Oil* | 11.1% | | |
| Eugenol | 3.0% | | |

*D-α-Tocopherol polyethylene glycol 1000 succinate
ZOE: zinc oxide eugenol

Powder was mixed with water at a ratio from 3:1 to 6:1. Additional loading of Eugenol was easily accomplished. The paste was exposed to water in tooth (or hot water bath) and then sets.
Liquid to be Mixed with a Powder
Vitamin E is insoluble in water and is high viscosity liquid. This interferes with the setting process of MTA. Therefore, a solution of D-α-Tocopherol polyethylene glycol 1000 succinate was mixed with water, stirred and warmed in the following ratio in Table 3:

TABLE 5
Vitamin E liquid composition

| Ingredient | Liquid |
|---|---|
| Water | 90% |
| DAE | 10% |

The powder (Table 3 or similar) was mixed with the liquid at a ratio of from 3:1 to 6:1. Additional loading of DAE is easily accomplished.

### Working and Setting Times

Liquid Powder—Ibuprofen powder:water 5:1

Working time>5 minutes

Set time <50 minutes

### Paste Eugenol—No mix

Working time>1 hour

Set time in hot water bath<3 hours

Liquid Powder—DAE (liquid) Cement (powder) powder:liquid 6:1

Working time>5 minutes

Set time<30 minutes

The composition of this invention has optimum working and setting times. Working time is measured according to Dental Standards IS09917 or ISO 6876 (water-based dental cements) and is the period of time measured from the initial mixing of the ingredients to the point when the material begins to harden—the material can be manipulated during this time with no adverse effect on the properties of the material. The net setting time is also measured according to Dental Standards ISO-9917 and is the period of time measured from the end of mixing of the ingredients to the point when the material sets. More particularly, the net setting time is measured by casting the material in a mold. After the mixing has been completed, the indenter device is vertically lowered onto the surface of the cement and it is allowed to remain there for 5 seconds. A trial run is carried out to determine the approximate setting time, repeating the indentations at 30 second intervals until the needle fails to make a complete circular indentation in the cement, when viewed using 2× magnification. The needle is cleaned, if necessary, between indentations. The process is repeated, starting the indentation at 30 seconds before the approximate setting time thus determined, making indentations at 10 second intervals. The net setting time is recorded as the time elapsed between the end of mixing and the time when the needle fails to make a complete circular indentation in the cement. A similar testing procedure, ISO 6876 (root canal sealers) can be used for measuring the working and setting times of the composition.

In general, the compositions of this invention have a working time in the range of about five (5) minutes to about sixty (60) minutes. The exact working time period of the composition depends on its specific formulation. As discussed above, different formulations can be used for root canal apicoectomies, apexification, perforation repair, obturation, pulpotomies, pulp-capping, cavity liners, root-end resorption repair, and root canal sealing. The final setting time is generally within the range of about ninety (90) minutes to about twelve (12) hours. This shortened working time allows the dental practitioner to handle and place the material more effectively. The clinician can fill or repair the root canal and see the material begin to harden and form a rock-like substance. The clinician is better able to work and shape the material. After the clinician applies the material to the targeted area, it remains in place. The material has good consistency and does not migrate away from the area. This allows a clinician to clean-up a site by rinsing when a surgical or vital pulp therapy procedure is performed, and blood is present. Furthermore, additional dental material, such as a restorative composite, can be placed over the root canal filling/sealing material as it begins to set. Used for pulp-capping or cavity liner procedures, the placed root canal filling/sealing material bonds to the root dentin and, preferably, to any other materials (for example, gutta-percha or dental composite) being used to fill the root canal or treat the vital pulp. As the root canal filling/sealing composition sets and hardens, it provides a solid barrier to bacterial and fluid leakage in the root canal system. The fluid pathways between the root canal system and surrounding tissue are tightly sealed off. Furthermore, the root canal filling/sealing material is bactericidal.

Workers skilled in the art will appreciate that various modifications can be made to the illustrated embodiments and description herein without departing from the spirit and scope of the present invention. It is intended that all such modifications within the spirit and scope of the present invention be covered by the appended claims.

1. A method of reducing an inflammation of a pulp or a periodontal ligament, said method comprising the steps of:
(a) removing material from a pulp cavity and/or a root canal of a tooth;
(b) rinsing pulp chamber with an anti-inflammatory rinse solution
(c) irrigating the root canal with an anti-inflammatory rinse solution to remove residual bacteria and/or smear layer
(d) providing an anti-inflammatory filling composition comprising:
(i) at least 80% by weight of a premixed cement putty, said cement putty including at least one calcium silicate compound in the range of about 30% to about 90% by weight of said putty; and at least about 1% to about 50% of a liquid carrier including a water-soluble polymer and/or water-soluble oil; and
(ii) about 3 to about 15% by weight of an anti-inflammatory agent, the anti-inflammatory agent including eugenol, vitamin E or mixtures thereof; and
(e) introducing the anti-inflammatory filling composition into the cavity and/or root canal and allow the composition to harden.

2. The method of claim 1, wherein the anti-inflammatory rinse solution further includes at least one anti-inflammatory agent selected from the group consisting of tannoid, benzydamine, Vitamin E, Eugenol, saline water, phosphate buffer saline, natural phytochemical antioxidant, chalcone, curcuminoid, carotenoid, sambucus nigra, Echinacea purpurea, hydrocotyle asiatica, xylitol and mixtures thereof.

3. The method of claim 2, wherein the anti-inflammatory rinse solution further includes a mixture of chlorhexidine and EDTA.

4. The method of claim 1, wherein the anti-inflammatory rinse solution includes eugenol in a concentration range of about 1 to about 30% by weight.

5. The method of claim 1, wherein the cement putty further includes about 1 to about 40% of a filler material.

6. The method of claim 5, wherein the filler material includes bioglass.

7. The method of claim 6, wherein the bioglass includes 35-55% by weight SiO₂; 12-35% by weight Na₂O; 10-32% by weight CaO, and 3-9% by weight P₂O₅.

8. The method of claim 5, wherein the filler material has a particle size of about 0.25 microns to about 120 microns

9. The method of claim 1, wherein the water-soluble polymer is selected from the group consisting of polyvinyl alcohols, polyvinyl pyrrolidine, poly vinyl acetates, and mixtures thereof.

10. The method of claim 1, wherein the water-soluble oil is D-α-Tocopherol polyethylene glycol 1000 succinate.

11. The method of claim 1, wherein the liquid carrier is a poly(alkylene glycol).

12. The method of claim 10, wherein the poly(alkylene glycol) is present in an amount of from 15% to 45% of the composition.

13. The method of claim 1, wherein the calcium silicate includes a mixture of tricalcium silicate and dicalcium silicate particles.

14. An anti-inflammatory endodontic filling composition comprising:
(a) at least 80 percent by weight of a premixed cement putty, said cement putty including at least one calcium silicate compound in the range of about 30% to about 90% by weight of said putty; and at least about 1% to about 50% of a liquid carrier including a water-soluble polymer and/or water-soluble oil; and
(b) about 1 to about 20% by weight of an anti-inflammatory agent including Eugenol, Vitamin E, or mixtures thereof.

15. The anti-inflammatory endodontic filling composition, according to claim 14, wherein the cement putty further includes about 1 to about 40% of a filler composition.

16. The anti-inflammatory endodontic filling material, according to claim 15, wherein the filler material includes bioglass.

17. The anti-inflammatory endodontic filling composition, according to claim 16, wherein the bioglass includes 35-55% by weight SiO₂; 12-35% by weight Na₂O; 10-32% by weight CaO, and 3-9% by weight P₂O₅.

18. The anti-inflammatory endodontic filling composition, according to claim 15, wherein the filler material has a particle size of about 0.25 microns to about 120 microns.

19. The anti-inflammatory endodontic filling composition, according to claim 14, wherein the water-soluble polymer is selected from the group consisting of polyvinyl alcohols, polyvinyl pyrrolidine, poly vinyl acetates, and mixtures thereof.

20. The anti-inflammatory endodontic filling composition, according to claim 14, wherein the water-soluble oil is D-α-Tocopherol polyethylene glycol 1000 succinate.

21. The anti-inflammatory endodontic filling composition, according to claim 14, wherein the liquid carrier is a poly(alkylene glycol).

22. The anti-inflammatory endodontic filling composition, according to claim 21, wherein the poly(alkylene glycol) is poly propylene glycol having a number average molecular weight in a range of from 1500 g/mol to 3000 g/mol.

23. The anti-inflammatory endodontic filling composition, according to claim 14, wherein the calcium silicate comprises a mixture of tricalcium silicate and dicalcium silicate particles.

24. An anti-inflammatory rinse solution for removing smear and disinfection in a root canal or pulp chamber, said solution comprising at least one anti-inflammatory agent.

25. The anti-inflammatory rinse solution, according to claim 24, wherein the anti-inflammatory agent is selected from the group consisting of tannoid, benzydamine, Vitamin E, eugenol, saline water, phosphate buffer saline, sodium hypochlorite, natural phytochemical antioxidant, chalcone, curcuminoid, carotenoid, sambucus nigra, Echinacea purpurea, hydrocotyle asiatica, xylitol and a mixture thereof.

26. The anti-inflammatory rinse solution according to claim 25, wherein the anti-inflammatory agent is eugenol.

27. The anti-inflammatory rinse solution according to claim 24, wherein the anti-inflammatory agent is present in concentration range of about 2 to about 25% by weight.

28. The anti-inflammatory rinse solution according to claim 24, wherein the anti-inflammatory rinse solution further includes a mixture of chlorhexidine and EDTA.
